(19) United States
(12) Patent Application Publication
(10) Pub. No.: US 2023/0371940 A1
(43) Pub. Date: Nov. 23, 2023

(54) JOINT DISTRACTION DEVICE

(71) Applicant: NEW YORK UNIVERSITY, New York, NY (US)

(72) Inventor: Andrew Rokito, New York, NY (US)

(21) Appl. No.: 18/247,155

(22) PCT Filed: Sep. 30, 2021
(86) PCT No.: PCT/US21/52923

§ 371 Date: Mar. 29, 2023

Related U.S. Application Data
(60) Provisional application No. 63/085,382, filed on Sep. 30, 2020.

(51) Int. Cl. A61B 17/02; A61B 46/20

(52) U.S. Cl. CPC A61B 17/025; A61B 46/20; A61B 2046/201; A61B 2046/236

(57) ABSTRACT

The present invention provides a method and apparatus for joint distraction during surgical applications. In one embodiment, the present invention provides a method and apparatus for joint distraction during any arthroscopic surgery including but not limited to a shoulder stabilization surgery, hip surgery, etc.

### CROSS-REFERENCE TO RELATED APPLICATIONS

This application claims priority to U.S. Provisional Patent Application No. 63/085,382 filed Sep. 30, 2020, the contents of which are incorporated by reference herein in its entirety.

### BACKGROUND OF THE INVENTION

Arthroscopy is a minimally invasive surgical procedure used in the examination and treatment of joint disease or damage. In order to provide access to a joint space for a diagnostic or therapeutic procedure, the joint often must be distracted. Distraction is the term used to define a combination of traction and distention used to separate the joint, which in the case of a hip joint, allows the surgeon to access the central compartment. During arthroscopic shoulder surgery, distraction of the humeral head away from the glenoid fossa is necessary. This is especially true during shoulder stabilization/Bankart repair procedures. Distraction is needed to gain access to the inferior quadrant of the joint. Without proper joint distraction, the surgeon's ability to visualize and correct the pathology associated with shoulder instability is impaired. Current commercially available joint distraction devices include 3-point fixation pulley systems and hydraulic powered lifts and arm holders. These devices are cumbersome, complicated to use, require specialized arm sleeves, are time-consuming to set-up in advance during patient positioning, and are expensive.

Thus, there is a need in the art for a distraction device that is easy to use, disposable and doesn't require the surgeon to plan in advance. The present invention meets this need.

### SUMMARY OF THE INVENTION

In one aspect the present invention provides a joint distraction device for use in an arthroscopy surgery, comprising: a drape configured for covering at least a portion of a patient during surgery, the drape having a top surface and a bottom surface and is configured to be disposed above the patient when in use during surgery, wherein the drape comprises an opening configured to accept a limb, and an inflatable member, wherein the inflatable member is positioned adjacent to the opening, and is inflated by a fluid from a deflated state to an inflated state. In one embodiment, the inflatable member is fluidly connected to a pump through an inlet port and is configured to allow fluid in to inflate the inflatable member. In one embodiment, the pump is a manual pump. In one embodiment, the pump is an electric pump. In one embodiment, the device further comprises a pouch configured to collect and/or retain any fluid from a surgical site. In one embodiment, the pouch is positioned on the top surface of the drape. In one embodiment, the pouch comprises a drainage port configured to allow drainage of any fluids collected during the surgery. In one embodiment, the device further comprises a pressure regulator. In one embodiment, the inflatable member is inflated with a liquid medium selected from the group consisting of water, saline, and combinations thereof. In one embodiment, the inflatable member is inflated with a gaseous medium. In one embodiment, the device is disposable.

In one aspect, the present invention provides a method for distracting a joint during arthroscopic surgery, the method comprising: providing a joint distraction device comprising a drape having a top surface, a bottom surface, an opening configured to accept a limb, and an inflatable member, wherein the inflatable member is positioned adjacent to the opening, and is inflated by a fluid from a deflated state to an inflated state; applying a force to a body part so as to distract the joint and position the joint distraction device in a desired position; inflating the inflatable member; and reducing the force applied to the body part so that the joint is supported on the inflatable member. In one embodiment, the inflatable member is fluidly connected to a pump through an inlet port and is configured to allow fluid in to inflate the inflatable member. In one embodiment, the pump is a manual pump. In one embodiment, the pump is an electric pump. In one embodiment, the device further comprises a pouch configured to collect and/or retain any fluid from a surgical site. In one embodiment, the pouch is positioned on the top surface of the drape and comprises a drainage port configured to allow drainage of any fluids collected during the surgery. In one embodiment, the device further comprises a pressure regulator. In one embodiment, the inflatable member is inflated with a liquid medium selected from the group consisting of water, saline, and combinations thereof. In one embodiment, the inflatable member is inflated with a gaseous medium.

### BRIEF DESCRIPTION OF THE DRAWINGS

The following detailed description of invention will be better understood when read in conjunction with the appended drawings. It should be understood, however, that the invention is not limited to the precise arrangements and instrumentalities of the embodiments shown in the drawings.

FIG. 1A through FIG. 1B depicts a perspective view of an exemplary joint distraction device of the present invention. FIG. 1A depicts an exemplary joint distraction device with the inflatable member in an inflated state. FIG. 1B depicts a perspective view of the inflatable member in a deflated state.

FIG. 2 depicts a side perspective view of an exemplary joint distraction device of the present invention.

FIG. 3 depicts another exemplary joint distraction device of the present invention.

FIG. 4 is a flowchart depicting an exemplary method of distracting a joint during an arthroscopic surgery.

### DETAILED DESCRIPTION

It is to be understood that the figures and descriptions of the present invention have been simplified to illustrate elements that are relevant for a clear understanding of the present invention, while eliminating, for the purpose of clarity many other elements found in the field of joint distraction devices. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Joint Distraction Device

The present invention provides a joint distraction device configured to provide visualization and instrumentation during arthroscopic surgery. In one embodiment, the joint distraction device described herein is configured to distract and maintain distraction of a joint during arthroscopic surgery. In one embodiment, the joint distraction device is able to distract joints including but not limited to the hip, shoulder, ankle, wrist joint, etc. In one embodiment, the joint distraction device of the present invention provides an “on demand” and easy method of distracting the joint during an arthroscopic surgery. In one embodiment, the joint distraction device of the present invention does not require any pre-planning or setup by the user.

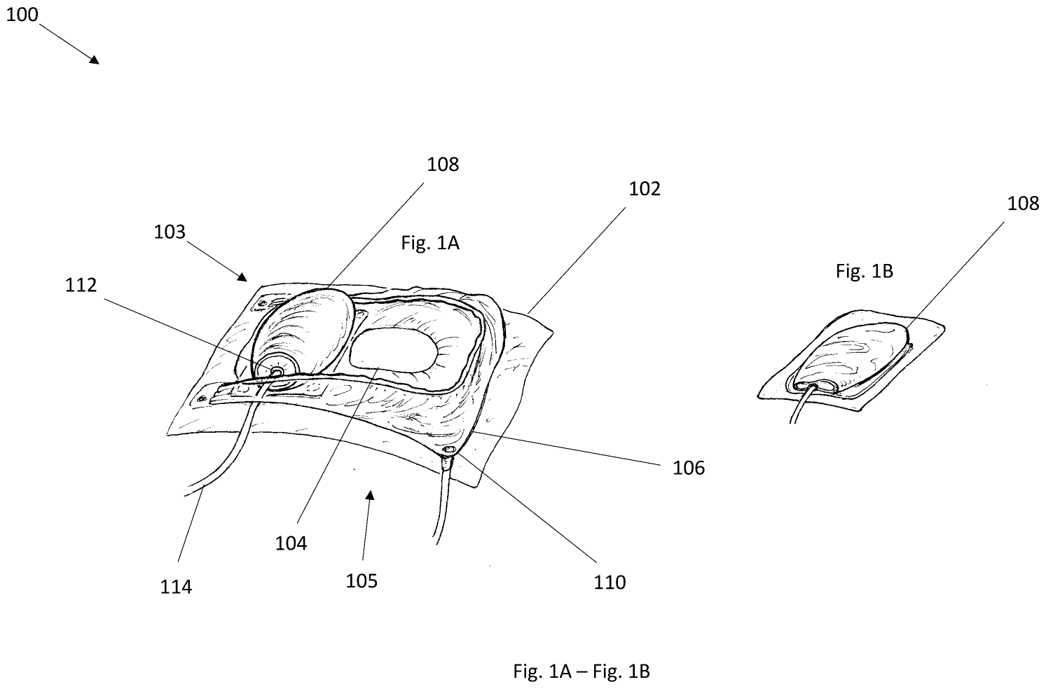

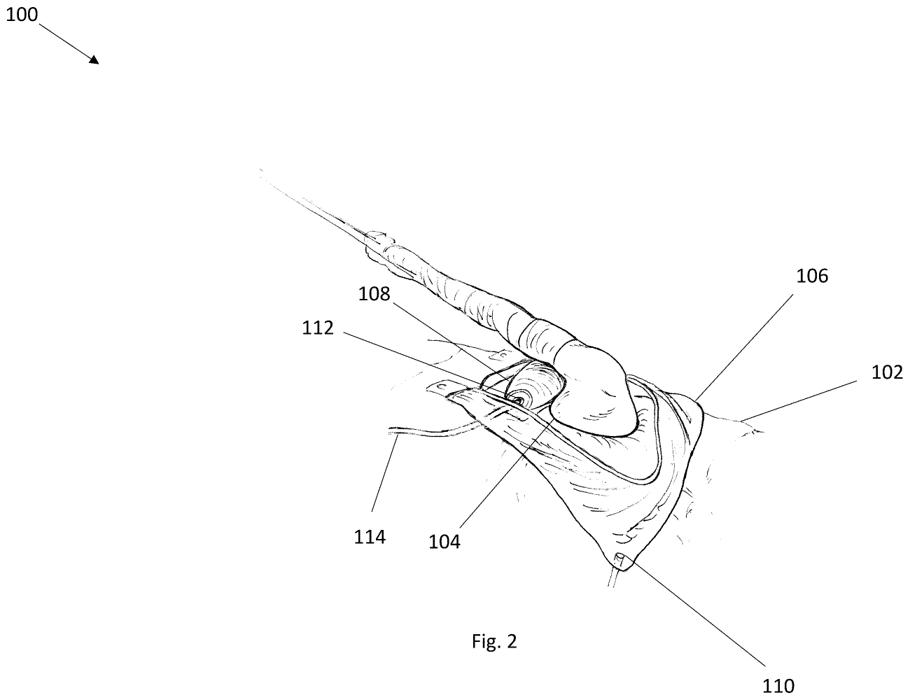

Referring now to FIG. 1A, FIG. 1B and FIG. 2, an exemplary joint distraction device 100 of the present invention is shown. Joint distraction device 100 comprises a drape 102 having a top surface 103, an opening 104, a bottom surface 105, a pouch 106, and an inflatable member 108.

In one embodiment, drape 102 may have any variety of shapes known to one skilled in the art including but not limited to rectangular, square, circular, oval, etc. In one embodiment, drape 102 may have any size known to one skilled in the art. In one exemplary embodiment, drape 102 may be sized to cover at least a portion of a patient's body. In one embodiment, drape 102 may be sized to cover a part of the patient body, portion of the operating table and associated wires, cables, trays, tools, and instruments, including but not limited to 3D radiographic equipment, which is used during the course of a sterile surgical procedure. In one embodiment, drape 102 may be made from any material known to one skilled in the art including but not limited to plastics, polymers, etc. In one embodiment, drape 102 may be made from a transparent or translucent material. In one embodiment, drape 102 may comprise nonwoven layers, adhesive layers, film layers, magnetic layers, etc. In one embodiment, drape 102 may be made from a material that is absorbent. In one embodiment, drape 102 may be chemically treated to achieve desired absorbency properties. In one embodiment, drape 102 may be attached to the patient by any means known to one skilled in the art including but not limited to adhesives. In one embodiment, drape 102 may be made from a flexible material.

In one embodiment, drape 102 may be made from one or more layers. In one embodiment, if drape 102 is made from a plurality of layers, the layers may be laminated to one another by using conventional techniques known to those skilled in the art. For example, in accordance with one exemplary embodiment one or more layers may be thermally laminated to one or more layers using discrete bond points.

Opening 104 is configured to accept a limb of a patient to facilitate the surgical procedure. In one embodiment, opening 104 may have any shapes known to one skilled in the art, including but not limited to circular, oval, rectangular, etc. In one embodiment, opening 104 may comprise a flexible or elastic material that conforms to the patient's limb in such a manner that it prevents surgical and/or bodily fluids from leaking between patient's limb and opening 104.

Pouch 106 is positioned on top surface 103 of drape 102 and is configured to collect and/or retain fluid produced by a surgical procedure. In one embodiment, pouch 106 may be coupled to drape 102 in any suitable manner known to one skilled in the art including but not limited to adhesives, bonds, heat welds, laminates, and the like. In one embodiment, pouch 106 may have any suitable shape including but not limited to circular, oval, horseshoe, etc. In one embodiment, pouch 106 may be formed from any suitable fluid impervious material including but not limited to plastics, polymers, wax-coated fabrics, and the like. In one exemplary embodiment, pouch 106 may have a horseshoe shape. In one embodiment, pouch 106 is positioned as such to partially surround opening 104 or both opening 104 and inflatable member 108. In one embodiment, pouch 106 may be made from a transparent material, translucent material, or an opaque material.

In one embodiment, pouch 106 may comprise a drainage port 110 configured to facilitate the draining of any fluids collected during the surgical procedure. In one embodiment, drainage port 110 may facilitate the draining of the fluid by gravity. In one embodiment, drainage port 110 may be coupled to a suction device to facilitate draining of the fluid. Drainage port 110 may be any suitable fitting that is coupled to pouch 106 in any suitable manner. In one embodiment, pouch 106 may be filled with fluid until the completion of the surgical procedure, at such time pouch 106 may be removed and subsequently disposed.

Inflatable member 108 is positioned on top surface 103 of drape 102 and is positioned adjacent to opening 104. In one embodiment, inflatable member 108 is attached to top surface 103 of drape 102 by any suitable manner known to one skilled in the art including but not limited to adhesives. Inflatable member 108 comprises an inflation/deflation control mechanism. Inflation of inflatable member 108 through the inflation/deflation control mechanism allows a user (e.g., surgeon, nurse, or other medical personnel) to lift the patient's limb coming through opening 104 to an optimum height (see FIG. 2). As it would be understood by one skilled in the art, inflatable member 108 is placed adjacent to opening 104 as this would enable inflation of inflatable member 108 to lift and distract the joint easily and quickly in an on-demand fashion, where the height change is most effective in stably supporting a distracted joint.

Inflatable member 108 comprises an inlet port 112 into which air or other suitable fluid (liquid or gas) may be directed to inflate inflatable member 108. In one embodiment, a pump may be used to deliver fluid into inflatable member 108 to inflate inflatable member 108. In one embodiment, the pump may be power operated. In one embodiment, the pump is an electric pump. In one embodiment, the pump may be hand operated. In one exemplary embodiment, the pump may be a manual pump such as a squeeze ball. In one embodiment, the pump is fluidly connected to inflatable member 108 through a flexible tube 114. In one embodiment, the pump is provided with two one-way valves. The first one-way valve is operative to permit fluid to flow from the pump to the tube each time the pump is manually squeezed. However, this first one-way valve prevents a return flow of fluid from the tube into the pump. The second one-way valve permits fluid flow from the ambient environment into the pump as the pump resiliently expands from the squeezed condition to the expanded condition. This second one-way valve, however, prevents an outflow of fluid from the pump to the ambient environment.

Additionally, it is to be understood that any type of fluid may be inserted into inflatable member 108 in order to move inflatable member 108 from a deflated state to an inflated state, and the present invention is not limited to configurations in which only air is used. In one embodiment, inflatable member 108 may be inflated with a liquid medium including but not limited to water, saline, etc. In certain embodiments, fluid that may be used during the surgical procedure, such as arthroscopic fluid or irrigation fluid, is used to inflate inflatable member 108. For example, in one embodiment, flexible tube 114 is in fluidic communication with an arthroscopic fluid pump, such that a user can direct the arthroscopic fluid pump to deliver arthroscopic fluid to inflatable member 108 if deemed necessary to distract the joint. In one embodiment, fluid coming from the surgical site may be used to inflate inflatable member 108. In one embodiment, inflatable member 108 may be inflated with arthroscopic fluid runoff that are used around any other limb including but not limited to knee, elbow, shoulder wrist, foot, ankle, and hip. In one embodiment, inflatable member 108 may be inflated with a gaseous medium including but not limited to air. In one embodiment, inflatable member 108 may be inflated with a high viscosity fluid. This latter construction may be beneficial in the event of inflatable member 108 puncture as it would slow the pace of deflation. In one embodiment, a fluid could be used which changes viscosity when subject to changes in temperature, electrical charge, magnetic field, or other means.

In one embodiment, any other device may be used to introduce fluids into inflatable member 108. For example, a syringe-type device may be connected to inflatable member 108 and fluid may be directed therein in order to effect inflation of inflatable member 108. Alternatively, a compressed gas in a cylinder, such as a 12-gm disposable CO₂ cylinder, may be used in order to inflate inflatable member 108.

In one embodiment, inflation member 108 may further comprise a pressure regulator, including but not limited to a release valve to ensure that inflatable member 108 is not inflated beyond a maximum level. In one embodiment, inflatable member 108 may further comprise an alarm or other alert to advise the user that inflatable member 108 has been inflated beyond a pre-determined level.

In one embodiment, inflatable member 108 may have any shape known to one skilled in the art including but not limited to a cylindrical shape, a polygonal shape, etc. In one embodiment, inflatable member 108 may also be in the form of an are or other curvature (i.e., a geometry where one side has a greater curvature than the other side), or some other shape (e.g., U-shaped).

In one embodiment, inflatable member 108 may comprise one or more chambers. In one embodiment, the chambers may be capable of being separately inflated into the inflated state, so that various heights could be reached.

In one embodiment, the surface of inflatable member 108 can be textured (e.g., with dimples, ridges, etc.) or covered with another material (e.g., a coating or covering) so as to prevent slippage of inflatable member 108 when it is being used to support a limb. In one embodiment, only a portion of inflatable member 108 may be textured. In one embodiment, the majority of inflatable member's surface may be textured.

In one embodiment, inflatable member 108 is inflatable to varying thickness and firmness.

In one embodiment, joint distraction device 100 is a sterile, single-use and disposable device. In one embodiment, joint distraction device 100 can be sterilized using techniques known to one skilled in the art. In one embodiment, joint distraction device 100 may comprise an outer cover that may be removed for cleaning/sterilizing. In one embodiment, joint distraction device 100 may be packaged and shipped to a prospective user with inflatable member 108 in a deflated state.

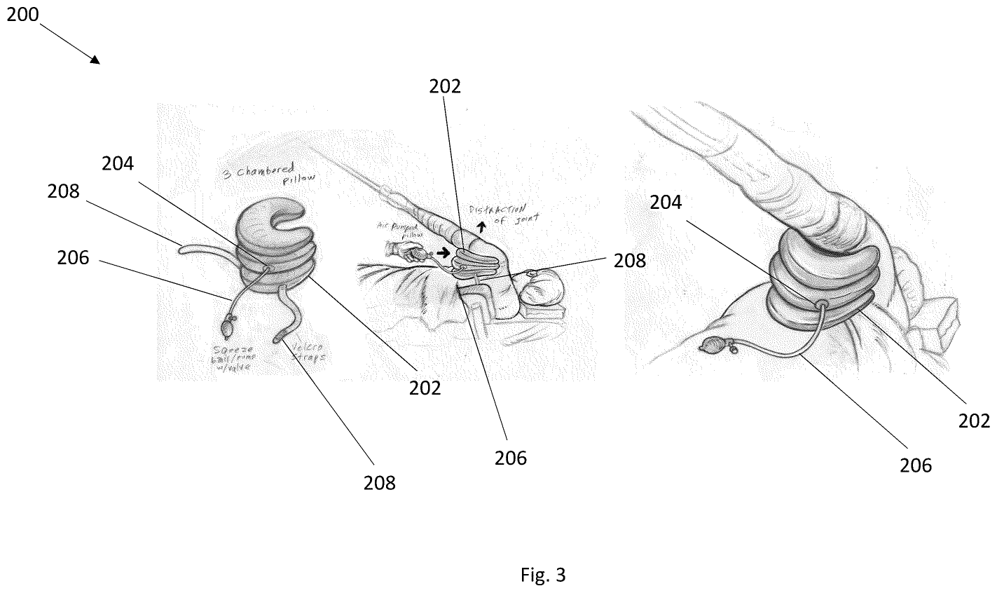

Referring now to FIG. 3, another exemplary joint distraction device 200 of the present invention is shown. In one embodiment, joint distraction device 200 comprises an inflatable pillow 202 having an inlet port 204 into which air or other fluid may be directed to inflate inflatable pillow 202.

Inflatable pillow 202 may comprise one or more chambers. In one embodiment, chambers may be capable of being separately inflated into the inflated state, so that various heights could be reached. In one embodiment, inflatable pillow 202 may have any shapes known to one skilled in the art including but not limited to oval, cylindrical, horseshoe, etc. In one embodiment, inflatable pillow 202 may be made from any applicable fluid impervious material known to one skilled in the art.

In one embodiment, a pump may be used to deliver fluid to inflatable pillow 202. In one embodiment, the pump may be power operated. In one embodiment, the pump may be hand operated. In one exemplary embodiment, the pump may be a manual pump such as a squeeze ball. In one embodiment, the pump is fluidly connected to pillow 202 through a flexible tube 206. In one embodiment, the pump is provided with two one-way valves. The first one-way valve is operative to permit fluid to flow from the pump to the tube each time the pump is manually squeezed. However, this first one-way valve prevents a return flow of fluid from the tube into the pump. The second one-way valve permits fluid flow from the ambient environment into the pump as the pump resiliently expands from the squeezed condition to the expanded condition. This second one-way valve, however, prevents an outflow of fluid from the pump to the ambient environment.

Additionally, it is to be understood that any type of fluid may be delivered into inflatable pillow 202 in order to move inflatable pillow 202 from a deflated state to an inflated state, and the present invention is not limited to configurations in which only air is used. In one embodiment, inflatable pillow 202 may be inflated with a liquid medium including but not limited to water, saline, etc. In certain embodiments, fluid that may be used during the surgical procedure, such as arthroscopic fluid or irrigation fluid, is used to inflate inflatable pillow 202. For example, in one embodiment, flexible tube 206 is in fluidic communication with an arthroscopic fluid pump, such that a user can direct the arthroscopic fluid pump to deliver arthroscopic fluid to inflatable pillow 202 if deemed necessary to distract the joint. In one embodiment, fluid coming from the surgical site may be used to inflate inflatable pillow 202. In one embodiment, inflatable pillow 202 may be inflated with arthroscopic fluid runoff that are used around any other limb including but not limited to knee, elbow, shoulder wrist, foot, ankle, and hip. In one embodiment, inflatable pillow 202 may be inflated with a gaseous medium including but not limited to air. In one embodiment, inflatable pillow 202 may be inflated with a high viscosity fluid. This latter construction may be beneficial in the event of inflatable pillow 202 puncture as it would slow the pace of deflation. In one embodiment, a fluid could be used which changes viscosity when subject to changes in temperature, electrical charge, magnetic field, or other means.

In one embodiment, any other device may be used to introduce fluids into inflatable pillow 202. For example, a syringe-type device may be connected to inflatable pillow 202 and fluid may be directed therein in order to effect inflation of inflatable pillow 202. Alternatively, a compressed gas in a cylinder, such as a 12 gm disposable CO₂ cylinder, may be used in order to inflate inflatable pillow 202.

In one embodiment, inflation pillow 202 may further comprise a pressure regulator, including but not limited to a release valve to ensure that inflatable pillow 202 is not inflated beyond a maximum level. In one embodiment, inflatable pillow 202 may further comprise an alarm or other alert to advise the user that inflatable pillow 202 has been inflated beyond a pre-determined level.

In one embodiment, the surface of inflatable pillow 202 can be textured (e.g., with dimples, ridges, etc.) or covered with another material (e.g., a coating or covering) so as to prevent slippage of inflatable pillow 202 when it is being used to support a limb. In one embodiment, only a portion of inflatable pillow 202 may be textured. In one embodiment, the majority of inflatable pillow's surface may be textured.

In one embodiment, inflatable pillow 202 is inflatable to varying thickness and firmness.

In one embodiment, joint distraction device 200 may further comprise a securement member 208 configured to fix device 200 in place while in use. In one embodiment, the securement member may have an adjustable length. In one embodiment, the securing member may be any mechanism known to one skilled in the art including but not limited to the use of a Velcro strap.

In one embodiment, joint distraction device 200 is a sterile, single-use and disposable device. In one embodiment, joint distraction device 200 can be sterilized using techniques known to one skilled in the art. In one embodiment, joint distraction device 200 may comprise an outer cover that may be removed for cleaning/sterilizing. In one embodiment, joint distraction device 200 may be packaged and shipped to a prospective user with inflatable member 208 in a deflated state.

### Method of Use

In one aspect, the present invention provides a method to distract and maintain distraction of a joint during surgery. In one embodiment, the method of the present invention is able to distract joints including but not limited to the hip, shoulder, knee, ankle, wrist joint and etc. In one embodiment, the method of present invention provides an “on demand” and easy method of distracting the joint during an arthroscopic surgery. In one embodiment, the method of present invention does not require any pre-planning or setup by the user.

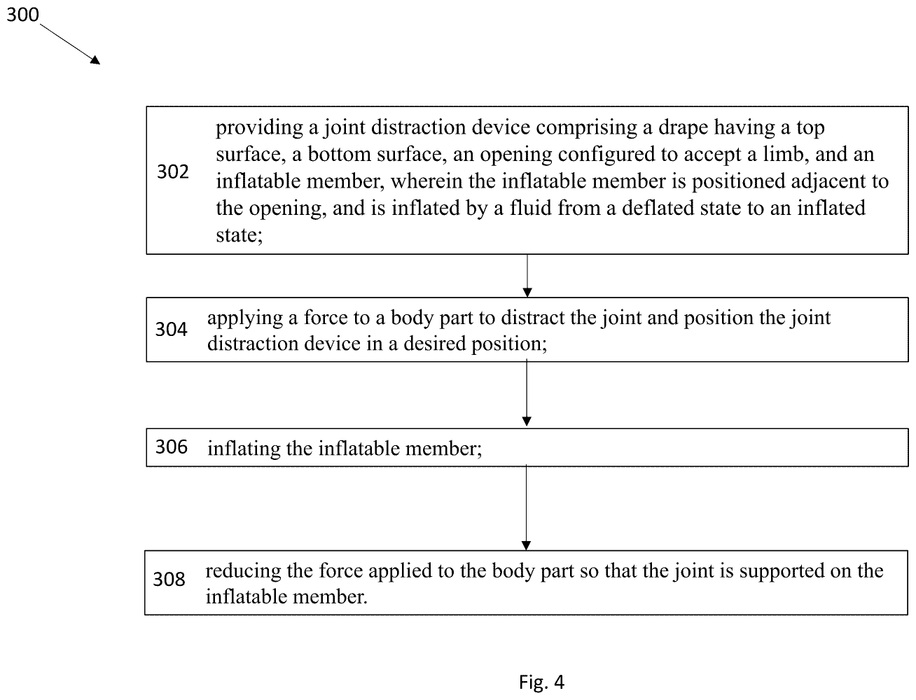

Referring now to FIG. 4, an exemplary method of distracting a joint 300 is shown. An exemplary method 300 begins with step 302, wherein a joint distraction device comprising a drape having a top surface, a bottom surface, an opening configured to accept a limb, and an inflatable member, wherein the inflatable member is positioned adjacent to the opening, and is inflated by a fluid from a deflated state to an inflated state is provided. In step 304, a force is applied to a body part so as to distract the joint and position the joint distraction device in a desired position. In one exemplary embodiment, during shoulder stabilization procedure, the patient is set-up in the traditional lateral decubitus position (position of choice for most surgeons for shoulder stabilization procedures) with a single traction pulley system. The joint distraction device is placed beneath the axilla of the operative arm. In step 306, the inflatable member is inflated. In one embodiment, the inflatable member may be inflated with a liquid medium including but not limited to water, saline and etc. In certain embodiments, fluid that may be used during the surgical procedure, such as arthroscopic fluid or irrigation fluid, is used to inflate the inflatable member. In one embodiment, the inflatable member may be inflated with a gaseous medium including but not limited to air. In step 308, the forces applied to the body part is reduced, so that the joint is supported on the inflatable member.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A joint distraction device for use in an arthroscopy surgery, comprising:
a drape configured for covering at least a portion of a patient during surgery, the drape having a top surface and a bottom surface and is configured to be disposed above the patient when in use during surgery, wherein the drape comprises an opening configured to accept a limb, and an inflatable member, wherein the inflatable member is positioned adjacent to the opening, and is inflated by a fluid from a deflated state to an inflated state.

2. The joint distraction device of claim 1, wherein the inflatable member is fluidly connected to a pump through an inlet port and is configured to allow fluid in to inflate the inflatable member.

3. The joint distraction device of claim 2, wherein the pump is a manual pump.

4. The joint distraction device of claim 2, wherein the pump is an electric pump.

5. The joint distraction device of claim 1, wherein the device further comprises a pouch configured to collect and/or retain any fluid from a surgical site.

6. The joint distraction device of claim 5, wherein the pouch is positioned on the top surface of the drape.

7. The joint distraction device of claim 5, wherein the pouch comprises a drainage port configured to allow drainage of any fluids collected during the surgery.

8. The joint distraction device of claim 1, wherein the device further comprises a pressure regulator.

9. The joint distraction device of claim 1, wherein the inflatable member is inflated with a liquid medium selected from the group consisting of water, saline, and combinations thereof.

10. The joint distraction device of claim 1, wherein the inflatable member is inflated with a gaseous medium.

11. The joint distraction device of claim 1, wherein the device is disposable.

12. A method for distracting a joint during arthroscopic surgery, the method comprising:
providing a joint distraction device comprising a drape having a top surface, a bottom surface, an opening configured to accept a limb, and an inflatable member, wherein the inflatable member is positioned adjacent to the opening, and is inflated by a fluid from a deflated state to an inflated state;
applying a force to a body part to distract the joint and position the joint distraction device in a desired position;
inflating the inflatable member; and
reducing the force applied to the body part so that the joint is supported on the inflatable member.

13. The method of claim 12, wherein the inflatable member is fluidly connected to a pump through an inlet port and is configured to allow fluid in to inflate the inflatable member.

14. The method of claim 13, wherein the pump is a manual pump.

15. The method of claim 13, wherein the pump is an electric pump.

16. The method of claim 12, wherein the device further comprises a pouch configured to collect and/or retain any fluid from a surgical site.

17. The method of claim 16, wherein the pouch is positioned on the top surface of the drape and comprises a drainage port configured to allow drainage of any fluids collected during the surgery.

18. The method of claim 12, wherein the device further comprises a pressure regulator.

19. The method of claim 12, wherein the inflatable member is inflated with a liquid medium selected from the group consisting of water, saline, and combinations thereof.

20. The method of claim 12, wherein the inflatable member is inflated with a gaseous medium.
